(19) United States
(12) Patent Application Publication
(10) Pub. No.: US 2020/0181205 A1
(43) Pub. Date: Jun. 11, 2020

(54) ENGINEERING OF MONOMERIC, RED-SHIFTED, AND BRIGHTER VARIANTS OF iRFP USING STRUCTURE-GUIDED MULTI-SITE MUTAGENESIS

(71) Applicants: Elad Firnberg, Baltimore, MD (US); Oliver C. Rogers, Baltimore, MD (US); Mark A. Verdecia, Baltimore, MD (US)

(72) Inventors: Elad Firnberg, Baltimore, MD (US); Oliver C. Rogers, Baltimore, MD (US); Mark A. Verdecia, Baltimore, MD (US)

(21) Appl. No.: 16/600,526

(22) Filed: Oct. 13, 2019

Related U.S. Application Data
(60) Provisional application No. 62/745,257, filed on Oct. 12, 2018.

(51) Int. Cl. C07K 14/195; C12N 9/22; C12N 9/88; C07K 19/00

(52) U.S. Cl. CPC C07K 14/195; C12N 9/22; C07K 2319/60; C07K 19/00; C12N 9/88

(57) ABSTRACT

The invention in at least one embodiment includes a method to convert a fluorescent protein into a modified fluorescent protein in a monomeric state where a dimerization interface is identified in a template protein in a homodimer state. One or more targeted mutation(s) are introduced at the dimerization interface to disrupt dimerization and favor the monomeric state. In another embodiment of the invention, one or more chromophore binding domains are identified in a template protein. One or more targeted mutations are introduced in the chromophore binding domain(s) to change the configuration of a biliverdin chromophore in the template protein from a 15Z anti configuration to a 15E anti configuration.

This patent application claims priority to U.S. Provisional Application 62/745,257, filed on Oct. 12, 2018, and incorporated herein by reference in its entirety.

### BACKGROUND

The present invention relates to systems and methods for the engineering of monomeric, red-shifted, and brighter variants of infrared fluorescent protein (iRFP) using structure-guided multi-site mutagenesis.

Genetically encoded fluorescent proteins (FPs) are life-science research tools. FPs based on green fluorescent protein (GFP) have been used for in vivo tissue imaging, cell tracing, and intracellular protein tracking. However, GFP-based FPs suffer several drawbacks including aggregation, photobleaching, and cytotoxicity. Additionally, tissue autofluorescence and absorbance at the wavelengths at which these FPs operate (350-530 nm) creates high background and a low signal to noise ratio¹. This limits their use to minimal surface cross-sections with a depth limit on single cell imaging of 0.05-0.1 mm and large tumor imaging up to 2.2 mm². Brain imaging studies are constrained to 1-2 mm in imaging depth³. To overcome this limitation, neuroscientists must use highly invasive surgical methods to image deeper parts of the brain. More recently, FPs functional in the far-red and near-infrared (NIR) spectral region (635-720 nm) have been developed that can provide higher penetration depth with lower background tissue absorbance and autofluorescence. However, several issues remain to be addressed for an optimal NIR FP reagent, including improved brightness and increased red-shifting. Perhaps the most notable limitation with currently available FPs are their oligomeric state, as all FPs in the NIR range exist primarily in a dimeric conformation. This property hinders the usefulness of these tools as it restricts gene fusion partners to those that can tolerate the dimeric FP state and may hinder the natural function of these proteins.

### SUMMARY OF THE INVENTION

The invention in at least one embodiment includes a method to convert a fluorescent protein into a modified fluorescent protein in a monomeric state where a dimerization interface is identified in a template protein in a homodimer state. One or more targeted mutation(s) are introduced at the dimerization interface to disrupt dimerization and favor the monomeric state. In another embodiment of the invention, one or more chromophore binding domains are identified in a template protein. One or more targeted mutations are introduced in the chromophore binding domain(s) to change the configuration of a biliverdin chromophore in the template protein from a 15Z anti configuration to a 15E anti configuration.

Another embodiment provides a monomeric protein including a biliverdin chromophore and a photosensory core domain (PCD). The PCD includes a chromophore binding domain (CBD) having a PAS (Per-Arnt-Sim) domain and a GAF (cGMP phosphodiesterase/adenylyl cyclase/FhlA) domain. The monomeric protein also includes one or more mutations, for example, a W309R mutation and a Q310A mutation.

### BRIEF DESCRIPTION OF DRAWINGS

The present invention is described with reference to the accompanying drawings. In the drawings, like reference numbers indicate identical or functionally similar elements.

FIG. 1 is a table illustrating homologous residues from several bacterial phytochrome proteins and their interaction with biliverdin groups in the photoconversion red (Pr) and far-red (Pfr) states according to an embodiment of the present invention.

FIG. 2 illustrates a homology model of iRFP shown in dimeric form according to an embodiment of the present invention.

FIG. 3A is a graph illustrating a gel filtration elution profile according to an embodiment of the present invention.

FIG. 3B is a graph illustrating a normalized fluorescence emission scans for the monomerized mutants according to an embodiment of the present invention.

FIG. 4A illustrates a crystal structure of the PAS (Per-Arnt-Sim) and GAF (cGMP phosphodiesterase/adenylyl cyclase/FhlA) domain from the multi-domain bacterial phytochrome photoreceptor (RpBphP2) according to an embodiment of the present invention.

FIG. 4B illustrates one monomer displaying the buried interface with critical dimerization residues F131/F132 shown in yellow and W309R/Q310A shown in green according to an embodiment of the present invention.

FIG. 4C illustrates A280 and fluorescence emission from gel filtration of iRFP wild-type and two monomeric mutants according to an embodiment of the present invention.

FIG. 4D is a graph illustrating normalized fluorescence emission spectra for monomerized mutants according to an embodiment of the present invention.

FIG. 4E illustrates on-plate NIR fluorescence imaged on the LI-COR Odyssey imager according to an embodiment of the present invention.

FIG. 5 illustrates an agarose DNA gel of PFunkel mutagenesis library reaction according to an embodiment of the present invention.

FIG. 6A illustrates an IVIS Lumina scan of an iRFP PFunkel library transformed into BL21 DE3 cells scanned at (Left) Ex: 680/Em:790 and at (Right) Ex:740/Em:790 with the yellow circle highlighting the colony originally expressing mRhubarb719 according to an embodiment of the present invention.

FIG. 6B illustrates an IVIS Lumina scan of BL21 cells streaked on an LB plate expressing mRhubarb713 (plate, upper-right), an unnamed, non-redshifted mutant with improved expression (plate, upper-left) and mRhubarb719 (plate, bottom) scanned at (Left) Ex: 680/Em:790 and at (Right) Ex:740/Em:790 according to an embodiment of the present invention.

FIG. 7 is a table illustrating spectral properties of iRFP variants according to an embodiment of the present invention.

FIG. 8 illustrates tables with excitation (dark colors) and emission (light colors) spectra for iRFP (blue) versus (A) mRhubarb713 (red), (B) mRhubarb719 (red), and (C) mRhubarb720 (red) according to an embodiment of the present invention.

FIG. 9 illustrates a table with emission spectra for all red-shifted or brighter mutants of iRFP in BL21 E. coli according to an embodiment of the present invention.

FIG. 10 is a flow diagram illustrating a method for the engineering of monomeric, red-shifted, and brighter variants of iRFP using structure-guided multi-site mutagenesis according to an embodiment of the present invention.

FIG. 11 is a flow diagram illustrating a method for the engineering of monomeric, red-shifted, and brighter variants of iRFP using structure-guided multi-site mutagenesis according to another embodiment of the present invention.

FIG. 12 illustrates a monomeric protein according to an embodiment of the present invention.

FIG. 13 illustrates a monomeric protein according to another embodiment of the present invention.

FIG. 14 illustrates a protein according to an embodiment of the present invention.

### DETAILED DESCRIPTION

Exemplary, non-limiting, embodiments of the present invention are discussed in detail below. While specific configurations are discussed to provide a clear understanding, it should be understood that the disclosed configurations are provided for illustration purposes only. A person of ordinary skill in the art will recognize that other configurations may be used without departing from the spirit and scope of the invention.

Far-red and near-infrared fluorescent proteins (FPs) can enable in vivo tissue imaging with greater depth and clarity compared to FPs in the visible spectrum due to reduced light absorbance and scatter by tissues. An embodiment of the invention provides a monomeric variant of iRFP, termed mRhubarb713, and subsequently uses a targeted and expansive multi-site mutagenesis approach to screen for variants with red-shifted spectral activity. Two monomeric variants are provided, deemed mRhubarb719 and mRhubarb720, with red-shifted spectra and increased quantum yield compared to iRFP. These tools may enable improved in vivo imaging studies with a genetically encoded reporter.

At least one embodiment of the invention improves upon FPs by using a structure-guided combinatorial mutagenesis strategy to engineer a monomeric FP with increased brightness and red-shifted spectra. Biliverdin (BV)-based fluorescent proteins, such as those of the bacterial phytochrome protein (BphP) family, can be useful templates for in vivo imaging applications. BV is an abundant heme metabolic product in many species including insects, plants, and mammals and does not need to be provided exogenously. Additionally, BV fluorescence may lack cytotoxicity unlike GFP-family FPs^{4, 5}. The NIR spectral properties of BV-based BphPs may be highly advantageous for deep tissue imaging. Light absorption and scatter from hemoglobin, deoxyhemoglobin, water, and lipids can reach a minimum in the 700-900 nm NIR window⁴. Light at these wavelengths can have greater incoming and outgoing tissue penetration depth compared to light in the visible spectrum. For these reasons, NIR FPs engineered for imaging purposes can utilize this chromophore as an endogenous fluorophore.

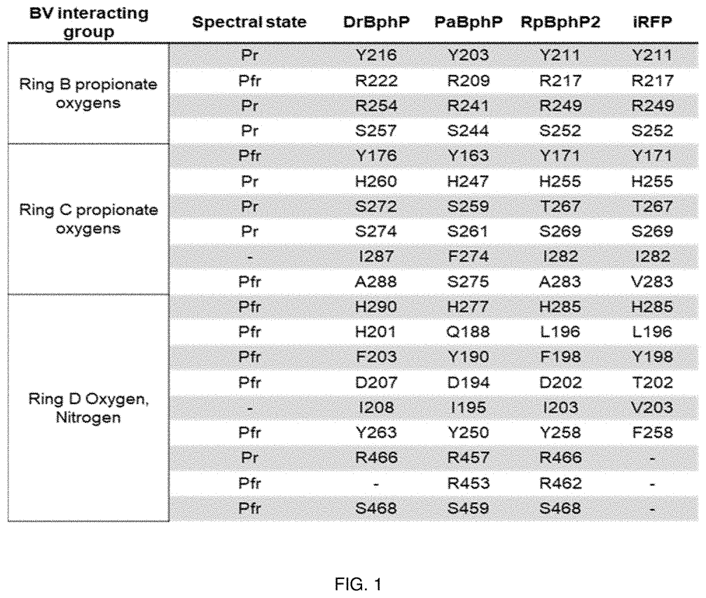

The BphP family of light-responsive proteins can share common domain architecture consisting of a photosensory core domain (PCD) and a histidine kinase (HK) output effector domain involved in downstream signaling. The PCD can be composed of three distinct domains, PAS (Per-Arnt-Sim), GAF (cGMP phosphodiesterase/adenylyl cyclase/FhlA) and PHY (phytochrome). In at least one embodiment, BphPs utilize biliverdin IXa (BV), a linear tetrapyrrole bilin which is a product of the oxidation of heme by heme oxygenase, as a chromophore. PAS and GAF can comprise the chromophore binding domain (CBD), which can catalyze the covalent attachment of BV to a PAS domain Cys residue via a thioester bond. The chromophore can cycle between a red-absorbing Pr state with absorption in the 680-710 nm range and a far-red absorbing Pfr state in the 740-760 nm range. In at least one embodiment, the Pr to Pfr photoconversion mechanism may involves an isomerization process about the C15=C16 double bond between rings C and D of BV, changing its configuration from 15Z anti to 15E anti^{7,8}. Additionally, a rotation of the entire chromophore may alter the interactions with several residues in the CBD (see FIG. 1)⁵. Phytochromes such as RpBph2 and DrBphP can have a Pr ground state that can photoconvert to Pfr upon illumination with red light. The bathy phytochromes, such as PaBphP, can have a Pfr ground state that can photoconvert to Pr upon illumination with far-red light. Return to the ground state may occur slowly via dark reversion or rapidly via illumination with far red or red light^{8,10}.

In at least one embodiment of the invention, iRFP is used as a template, which is a FP derived from the multi-domain bacterial phytochrome photoreceptor RpBphP2 from the purple non-sulfur bacteria Rhodopseudomonas palustris⁶. iRFP can be composed of the N-terminal 316 amino acids of RpBphP2, which can retain the PAS and GAF domains while excluding PHY and HK. Due to specific mutations introduced to the protein and its lack of the PHY domain, iRFP can be locked in the Pr state and may not photoconvert. Random mutagenesis on the original template to increase fluorescent brightness can result in 13 substitutions in the wild-type sequence. iRFP can have an excitation maximum of 690 nm and an emission maximum of 713 nm with a quantum yield of 6.3% and photostability of 960 s. A further engineered variant, iRFP720, with excitation/emission of 702/720 nm can have four additional mutations with slightly lower quantum yield (6.0%) and photostability (490 s)⁷. Other variants, such as mIFP, miRFP703, miRFP709 can be developed which have been shown to be monomeric in nature. However, these proteins can all either blue-shifted compared to iRFP or suffer notable decreases in molecular brightness.

In order to create a monomeric, red-shifted variant of iRFP with comparable brightness, a high-throughput combinatorial library screening approach using PFunkel multi-site mutagenesis can be used. The PFunkel technique can enable rapid construction of large user-defined combinatorial libraries in plasmid format. Information from BphP-family crystal structures, mutagenesis studies, and in silico models can be used to guide the selection of target residues that could modulate BV conformation and bias it towards a 15E Pfr-like fluorescent state. In at least one embodiment, residues targeted for mutagenesis included those determined to interact with the chromophore, located proximal to the chromophore-binding pocket, or shown to affect photoconversion dynamics in related BphPs.

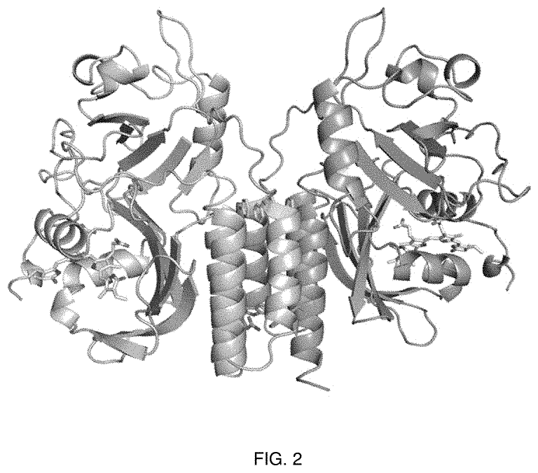

Given its ability to fluoresce in the NIR window in vivo, iRFP can be a useful template from which to design new tools to monitor a variety of cellular processes. iRFP can be a dimer in solution based on the crystal structure of RpBphP2⁷. In at least one embodiment, the oligomerization state of iRFP is assessed via gel filtration chromatography and found that the protein elutes as two distinct peaks, suggesting a mixed population or multiple stoichiometries. Thus, iRFP can exist as a heterogeneous mixture of two separate oligomerization states (FIG. 2) under conditions tested. This heterogeneity can complicate the interpretation of results from efforts to engineer activities into iRFP and disrupt structure-function relationships of a protein of interest when expressed as a fusion. FIG. 2 illustrates a homology model of iRFP shown in dimeric form according to an embodiment of the present invention. Mutations that can disrupt the dimer interface are shown in yellow (F131S/F132D) and green (W309R/Q310A). The BV chromophore is shown in the binding pocket.

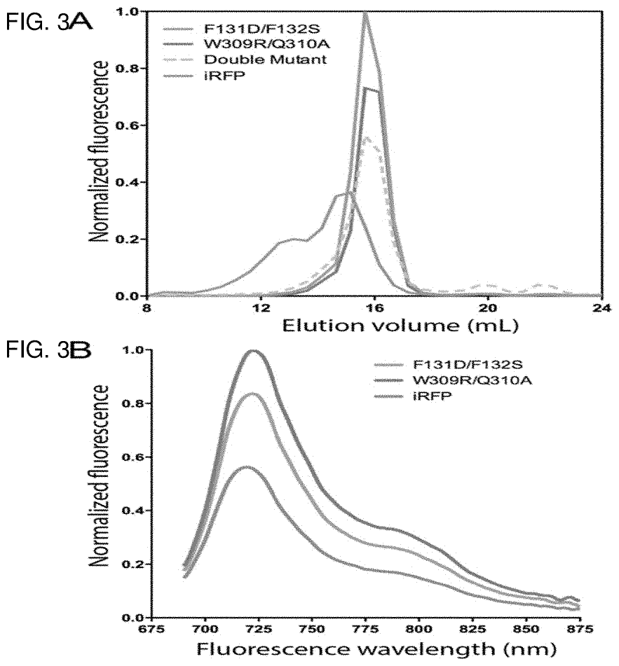

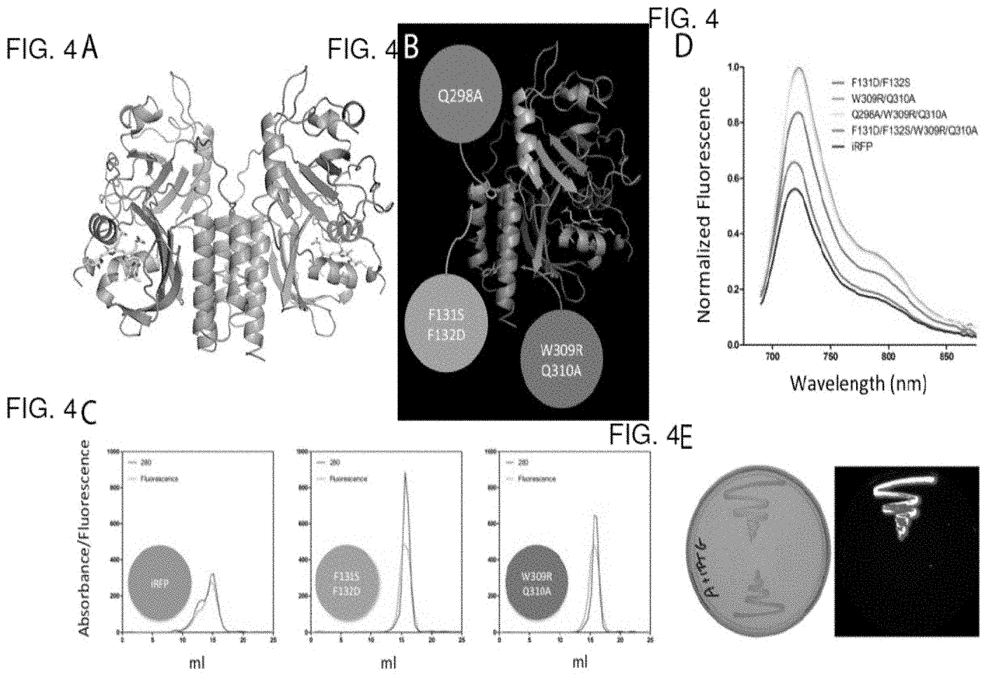

An atomic resolution homology model of iRFP bound to BV in the red-absorbing Pr state can be created using Modeller software. The homology model, as shown in FIG. 3A-B, can be based on the crystal structure of the chromophore-binding domain of RpBphP2 (PDB #4E04), with which iRFP may share 91% identity. This model can be used to guide a point mutation strategy to create an iRFP monomer (FIG. 3A-B). Analysis of the homodimer buried interface may show two pairs of residues F131/F132 and W309/Q310 that can contribute significant interactions to the interface. F131/F132 may consist of two Phe residues that pi-stack with their adjacent counterparts. In at least one embodiment, near the C-terminus, W309/Q310 sit at the bottom of the interface such that Trp residues stack together and the Gln reaches across the interface to form hydrogen-bond interactions with the helix of the other monomer. In one mutant, F131S and F132D, mutations may be introduced to remove the hydrophobicity and bulkiness of the segment while creating easily solvated charged interactions that would repulse the adjacent monomer. In a second mutant, W309R and Q310A, mutations may be introduced to create steric clash and abolish the charged interaction across the dimerization helices. Gel filtration can demonstrate that the F131S/F132D and W309R/Q310A mutants each elute as a single monomeric peak that is shifted towards a smaller apparent mass relative to the wild-type iRFP (FIG. 2). Both mutant proteins may be tested for BV binding and fluorescence and can be shown to be competent for both activities (FIG. 4A-E). Thus, these changes in the putative dimerization interface may produce the effect of shifting the oligomerization state from a mixed population to a monomeric state.

Both W309R/Q310A and F131S/F132D mutants can produce highly stable monomers with increased brightness compared to wild-type iRFP (FIG. 4A-E). Given its improved brightness and monomeric oligomeric state, the W309R/Q310A mutant is deemed as mRhubarb713, the first of three monomeric variants generated. The W309R/Q310A/F131S/F132D mutant can produce a monomer but may not be effectively brighter than the wild-type iRFP. Typically, oligomeric and dimeric proteins can be brighter than their monomeric counterparts due to more efficient folding and incorporation of the chromophore. For example, the red FP derived from Discoma coral DsRed was initially isolated as obligate oligomers, lost almost all fluorescence upon monomerization, and required extensive engineering to brighten⁸. In the case of iRFP, interactions between secondary structural elements of the CBD and the chromophore may be enhanced by monomerization resulting in decreased BV conformational freedom and increased fluorescence. Indeed, the native RpBphP2 can function by converting light induced conformational changes in BV into energy to modulate the function of PHY and HK domains⁹. The dimer may be used for facilitating this light induced work.

FIG. 4A illustrates a crystal structure of the PAS and GAF domain from RpBphP2 according to an embodiment of the present invention. The BV chromophore, shown in gray, can be covalently attached to the PAS domain Cys and cradled by the GAF domain within each identical monomer, shown in pink and green. The putative dimerization interface can comprise three helices forming a helix bundle that buries a significant surface comprised of both hydrophobic and charged residues. FIG. 4B illustrates one monomer displaying the buried interface with critical dimerization residues F131/F132 shown in yellow and W309R/Q310A shown in green according to an embodiment of the present invention. FIG. 4C illustrates A280 and fluorescence emission from gel filtration of iRFP wild-type and two monomeric mutants according to an embodiment of the present invention. FIG. 4D is a graph illustrating normalized fluorescence emission spectra for monomerized mutants according to an embodiment of the present invention. FIG. 4E illustrates on-plate NIR fluorescence imaged on the LI-COR Odyssey imager according to an embodiment of the present invention. Cells in the top streak express iRFP along with heme oxygenase while cells in the bottom streak express iRFP alone.

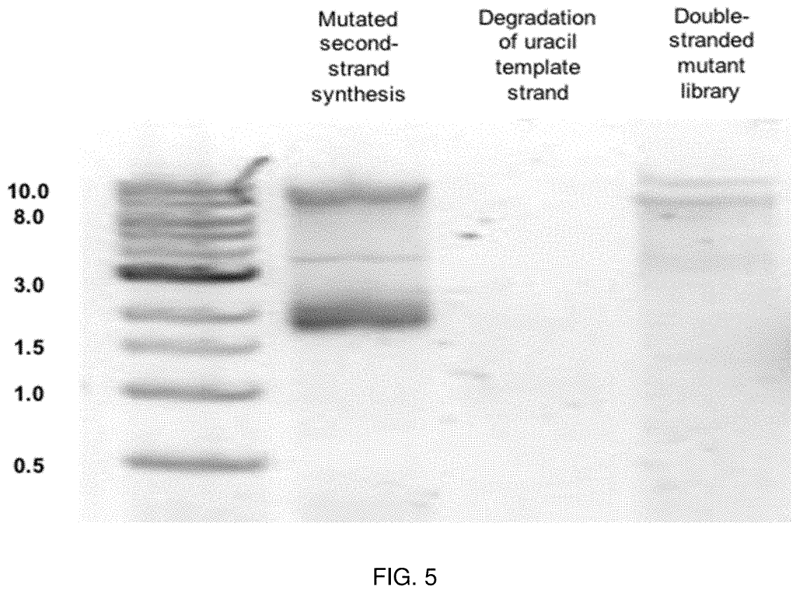

A second homology model representing iRFP bound to BV in the far-red-absorbing Pfr state can be generated based on the crystal structure of PaBphP (PDB #3C2W) in the Pfr state. Both model structures can be energy minimized and aligned for comparison. Several interactions between iRFP amino acid side chains in the CBD may differ between the Pr and Pfr models. These sites may provide a starting point for saturation mutagenesis in order to shift conformation towards the Pfr state. Based on these models, sites chosen for randomization can include V6, A7, R8, Q9, P10, Y171, F173, K193, Y198, T202, V203, R217, R249, T267, S269, L281, V283, and H285. The NNC codon can be chosen for randomization at each site, which may provide access to 15 amino acids while avoiding stop codons. In at least one embodiment, fifteen libraries were generated using PFunkel mutagenesis which introduced randomization in different combinations of the above positions. Efficiency of PFunkel reactions can be assessed by running each step of the reaction on an agarose gel (See FIG. 5). FIG. 5 illustrates an agarose DNA gel of PFunkel mutagenesis library reaction according to an embodiment of the present invention. Template dU-ssDNA is incubated with NNC primer library and Pfu polymerase to make a double stranded hetero-duplex (left). Using UDG, Exo I, and Exo III, the duplex is degraded and the template dU-ssDNA is degraded (middle). The single-stranded plasmid library is extended into its double-stranded product using a distal-binding primer (right).

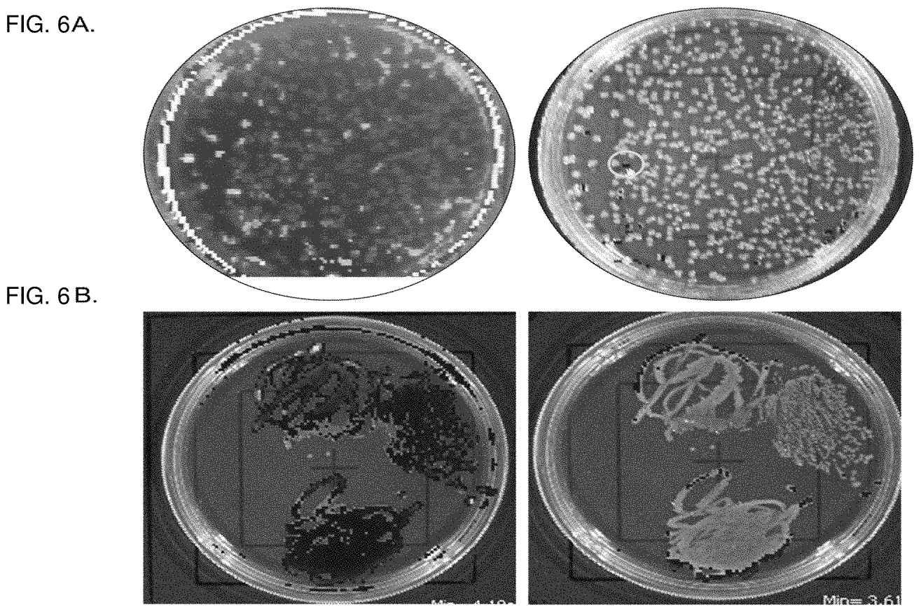

Library transformation yields can range from 10⁴ to 10⁶ colonies. Following plating on LB-agar bioassay dishes and NIR plate imaging, fluorescent colonies suspected of red-shifting can be selected, sequenced, and expressed for further analysis. A challenge that may be encountered with this screening approach is the difficulty in distinguishing colonies with enhanced brightness or expression (of which there were many) from colonies with truly red-shifted emission spectra (of which there were few, FIG. 6A-B). In at least one embodiment of the invention, a mutant from this screen, termed mRhubarb719 was identified harboring three mutations, Y198S, T202Y, V203I with an excitation peak of 700 nm, emission peak of 719 nm, quantum yield of 6.80% and relative molecular brightness of 92% compared to iRFP.

FIG. 6A illustrates an IVIS Lumina scan of an iRFP PFunkel library transformed into BL21 DE3 cells scanned at (Left) Ex: 680/Em:790 and at (Right) Ex:740/Em:790 with the yellow circle highlighting the colony originally expressing mRhubarb719 according to an embodiment of the present invention. FIG. 6B illustrates an IVIS Lumina scan of BL21 cells streaked on an LB plate expressing mRhubarb713 (plate, upper-right), an unnamed, non-redshifted mutant with improved expression (plate, upper-left) and mRhubarb719 (plate, bottom) scanned at (Left) Ex: 680/Em:790 and at (Right) Ex:740/Em:790 according to an embodiment of the present invention.

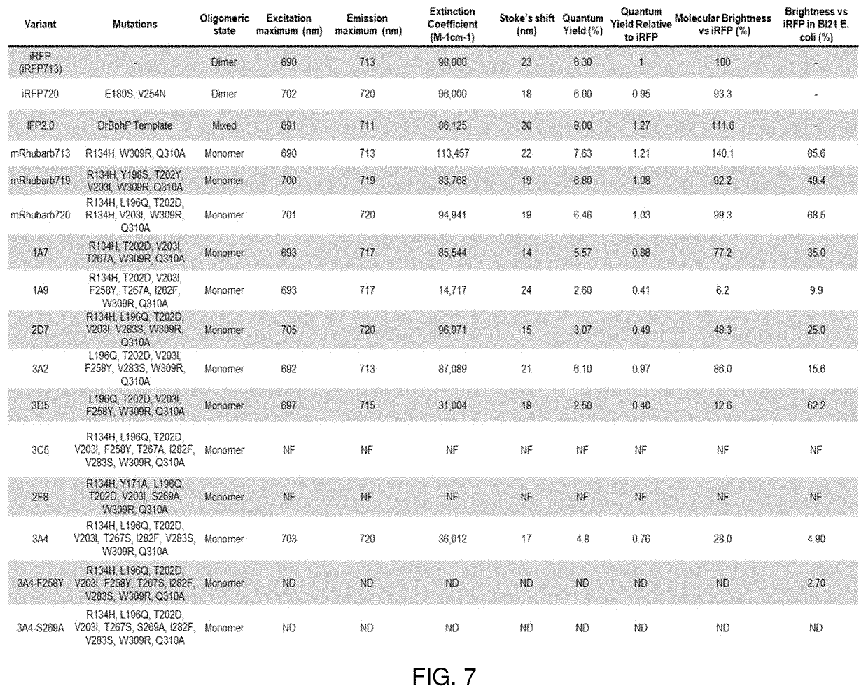

Due to the signal-to-noise challenges that may be encountered when screening large libraries on plates, a focused combinatorial mutagenesis approach can be utilized based on homologous residues previously shown to play critical roles in forming the PaBphP Pfr and Pr states (see FIG. 1). PaBphP can have a Pfr ground-state absorbance peak of 751 nm and may be crystallized in both dark Pfr and light Pr states^{5,10,11}. Mutagenesis studies have shown which residues play critical roles for stabilizing and destabilizing the Pfr state. Thus, a rational for this strategy may be to bias the chromophore conformation towards a fluorescent Pfr state by introducing mutations that stabilize the 15E conformation while destabilizing 15Z. In at least one embodiment, PFunkel multi-site mutagenesis is used to make a focused library of all 4096 possible combinations of 12 mutations useful for photoconversion dynamics. Individual clones may be grown in 96-well format and screened by spectral analysis in a Spectramax fluorescence platereader. Mutants displaying red-shifted emission spectra can be selected, sequenced and expressed for further analysis. This screen can identify 9 mutants with red-shifted spectral characteristics as shown in FIG. 7, one of which, deemed mRhubarb720 can have an excitation peak of 701 nm and an emission peak of 720 nm. This variant can also have a favorable quantum yield of 6.46% and brightness of 99% compared to iRFP making it the brightest, red-shifting mutant generated using the embodiments of the invention.

FIG. 7 is a table illustrating spectral properties of iRFP variants according to an embodiment of the present invention. NF (No Fluorescence) may denote that a clone was completely lacking fluorescence under all conditions tested. ND (Not Determinable) may denote that minimal (less than 10% of dimer) fluorescence was detected but the respective fluorescent parameter was likely not accurate.

Mutations Y171A and R249A may abolish fluorescence in all mutants. Mutations T202D and V203I, which can be reversions to the original RpBph2 sequence, may be found in all red-shifted variants. However, most of these may have reduced brightness. While D202 is noted as a residue for forming the Pfr state, high intensity fluorescence may only be noted in PaBphP once the homologous D194A mutation is introduced¹¹. Thus, the reduced brightness upon mutational reversion is consistent with previous findings.

The arm of the PHY domain may be involved in forming the Pfr state. Specifically, R453 and S459 in PaBphP (R462 and S468 in RpBphP2) may stabilize the flipping of the BV ring D. Mutations R453A and S459A may disrupt formation of the Pfr state¹¹. Therefore, reintroducing this portion of the PHY domain into iRFP may stabilize the Pfr state without inducing the signaling conformational change of the full-length phytochrome. In at least one embodiment, multiple red-shifted mutants are generated in which the PHY arm from PaBphP E431-S471 is fused to the N-terminus with a GGGGS linker and to the C-terminus with a (GGGGS)x2G linker. However, when expressed in E. coli under all conditions, these mutants may not express at amounts sufficient to characterize their fluorescence or may not express to detectable levels but may not be completely non-fluorescent. The fusion of PHY domain segment to either terminus of iRFP may yield unstable mutants incapable of proper folding and function.

To assess the in vivo properties of each mutant protein, each construct can be expressed in BL21 E. coli and the brightness in these cells may be compared. The iRFP template can yield the brightest transformed E. coli when compared to any of the other mutants including mRhubarb713, which can have notably improved molecular brightness in vitro. The discrepancy may suggest that dimeric structure of iRFP promotes stability and improved expression in cells. The molecular properties of each construct are not necessarily predictive of in vivo brightness, which may likely vary in different cell lines and organisms. Thus, prior to using these constructs in specific cell models, the optimal variant may be selected and expression conditions optimized.

Thus, three monomeric variants of iRFP, termed mRhubarb713, mRhubarb719, and mRhubarb720 can be developed, all of which can have either favorable brightness or red-shifting when compared to the dimeric iRFP. This can be enabled by two expansive multi-site combinatorial mutagenesis screens guided, rationally, by structural information of the protein. Of these two methods, the more randomized, high throughput approach can generate hundreds of thousands of iRFP mutants. This approach produced mRhubarb719. When a more narrowed, rational screen (which focused on stabilizing the Pfr state of the protein) is used, multiple red-shifted variants of iRFP may be characterized.

In at least one embodiment of the invention, the gene for iRFP is synthesized (Integrated DNA Technologies, Coralville, Iowa) and subcloned into the NcoI and BamHI restriction sites in the pHIS8-3 vector. All site-directed mutants of iRFP used for activity assays can be constructed using the QuikChange (Stratagene) protocol. Wild-type and mutants can be expressed using pHIS8-3 plasmid for oligomerization studies.

The construct used may consist of the pETDUET-1 vector (Novagen, Madison, Wis.) co-expressing the iRFP and heme oxygenase (for generation of the BV chromophore) genes from two separate T7/lacO promoters. The N-terminal residues of the iRFP gene can be MGSS-H₆-SQDP, which can include the 6×His tag and excess residues from the multiple cloning site. Additionally, the iRFP gene may contain a point mutation resulting in an R134H mutation which may not affect activity.

NiNTA purified iRFP mutants may be further purified and analyzed using a Superdex 200 26/60 gel filtration column (Amersham Biosciences, Little Chalfont, United Kingdom) equilibrated in 50 mM Tris-Cl (pH 8.0), 500 mM NaCl at 4° C. Peak fractions can be collected and dialyzed against 5 mM Tris-Cl (pH 8.0) and 100 mM NaCl, concentrated to 10 mg/ml using an Amicon Centricon 10 column (Millipore, Billerica, Mass., United States). All mutants may be compared to the elution volume of the dimeric Wt protein. All modeling of iRFP can be done using the RpBphP2 (PDB #4E04) entry in PyMol. All 3D structures can also be generated using PyMol.

pET-DUET-1-iRFP-HO plasmid ssDNA can be generated as previously described¹². PFunkel multi-site mutagenesis can be performed as previously described with minor modification¹³. In at least one embodiment of the invention, a 100 μL reaction is prepared in a 0.5 mL microtube containing 1×PfuTurbo Cx Hotstart DNA polymerase buffer, 5% DMSO, 0.2 mM dNTPs, 0.5 mM NAD+, 1 mM MgCl2, 140 nM phosphorylated oligos, and 4.6 nM ssDNA template. The reaction can be heated to 95° C. for 2 min, and 63.6° C. for 10 min. Then 0.025 U/uL PfuTurbo Cx Hotstart DNA polymerase and 2 U/uL Taq ligase, pre-activated by heating to 95° C. may be added to the reaction. Temperature cycling may proceed at 63.6° C. for 60 min, 45° C. for 15 min, 4° C. for 10 min, 95° C. for 2 min, and 45° C. for 15 min. Then 0.10 U/uL uracil DNA glycosylase, 0.29 U/uL exonuclease III, and 0.38 U/uL exonuclease I may be added to the reaction and incubation can continued at 37° C. for 60 min. Then 50 nM secondary phosphorylated oligo can be added to the reaction and cycled at 95° C. 2 min, 63.6° C. 30 min, and 45° C. 15 min. Aliquots may be withdrawn after first extension/ligation, strand degradation, and second strand extension/ligation steps to validate correct product formation on an agarose gel. The product DNA can be purified using the Nucleospin Gel and PCR clean-up kit (Macherey-Nagel, Duren, Germany). E. cloni EXPRESS BL21(DE3) cells (Lucigen, Middleton, Wis.) cells can be transformed with product DNA, plated on agar plates containing ampicillin 100 μg/mL, and grown at 37° C. overnight.

In at least one embodiment of the invention, fifteen combinatorial libraries are generated using PFunkel mutagenesis utilizing different combinations of mutagenic primers at the residues V6, A7, R8, Q9, P10, Y171, F173, K193, Y198, T202, V203, R217, R249, T267, S269, L281, V283, and H285. Libraries may be plated on LB-agar with 50 μM IPTG on bioassay dishes at a density of 10⁴-10⁷ CFU/plate via transformation of high efficiency E. cloni T7 EXPRESS BL21 (DE3) electrocompetent cells (Lucigen Corporation, Middleton, Wis.) according to manufacturer's instructions. After overnight colony growth and protein expression, plates may be imaged on the IVIS Lumina III instrument using an excitation wavelength of 700 nm and emission wavelengths of 720, 740, 760, and 780 nm. All colonies can be compared to the brightness of the wildtype iRFP. Colonies may be picked and grown up in LB containing ampicillin for further analysis.

PFunkel multi-site mutagenesis can be used to make a focused library making all possible combinations of Y171A, L196Q, F198Y, T202D, V203I, Y211A, R249A, H255A, F258Y, T267A, S269A, I282F, and A283S. Individual colonies can be seeded into 1 mL of 2×YT in 96 deep-well plates and grown overnight at 37° C. with shaking. Ten microliters of culture can be transferred to 1 mL of 2×YT in a 96 deep-well plate and incubated at 37° C. with shaking at 250 rpm until reaching an OD of 0.4-0.8. Expression may be induced by addition of IPTG to 500 μM and incubation can continue at 22° C. overnight with shaking at 250 rpm. Cultures may be pelleted at 4500×g for 10 min and the supernatant withdrawn. The pellets can be resuspended in 200 μL PBS and transferred to a 96 black-well plate. Emission spectra in culture format may be read on a Spectramax M2 platereader (Molecular Devices, Sunnyvale, Calif.) with excitation at 400 or 650 nm with a 695 nm cutoff and emission scan from 700-800 nm.

iRFP variants can be expressed in E. coli BL21(DE3) cells. A freshly transformed colony can be picked and grown in 100 mL LB with carbenicillin 100 μg/mL overnight at 37° C. with shaking, then 1/100^{th} volume can be used to inoculate LB media containing carbenicillin 100 μg/mL. The culture may be incubated at 37° C. shaking at 250 RPM until an OD600 of 0.4 to 0.8 was reached. Protein expression may be induced by addition of 500 μM IPTG and incubation at 22° C. shaking at 250 RPM for 16 hrs. The cells can be centrifuged at 4000×g for 20 minutes and lysed by resuspending the cell pellet in 1 mL BugBuster Master Mix protein extraction reagent (Novagen, Madison, Wis.) containing SIGMAFAST protease inhibitor cocktail (S8830, Sigma-Aldrich, Saint Louis, Mo.). The mixture can be incubated at room temperature for 30 minutes and centrifuged at 18,000×g for 30 minutes to remove insoluble material. The clarified lysate can be mixed with washed Hispure Ni-NTA resin (88221, Thermo Scientific), washed with PBS pH 7.4 containing 20 mM imidazole, and eluted in 1 mL PBS containing 250 mM imidazole. Protein concentration was may be determined using the Bradford assay as well as band densitometry and ImageJ software analysis. All protein preps can be run on SDS PAGE using Mini-PROTEAN TGX Pre-stained gels. Stocks of iRFP and all mutants can be stored at −80° C.

To characterize each mutant, purified variants can be diluted in PBS with 250 mM imidazole to a concentration of 0.6 mg/mL. This solution may be diluted two-fold serially multiple times to generate a concentration gradient. Samples can be first scanned for their emission maxima using an excitation wavelength of 400 nm and emission wavelengths of 670-770 nm. To determine the excitation maximum for each mutant, absorbance scans may be taken at wavelengths ranging from 600-750 nm. Relative quantum yield can be calculated by first determining the slopes of linear functions comparing the total integrated fluorescence of each mutant versus the absorbance at max at various protein concentrations¹⁴. These calculated slopes can be compared directly to iRFP. The extinction coefficient can be determined by plotting the absorbance at maximum versus the relative protein concentration and comparing directly to iRFP. The brightness relative to iRFP may be calculated as the product of the quantum yield and the extinction coefficient.

BL21 DE3 E. coli can be transformed with plasmids for all mutants displaying improved brightness or red-shifting compared to iRFP. Single colonies may be picked and inoculated overnight at 37° C. in 5 mL terrific broth medium shaking at 250 RPM with no IPTG added. Cells can be centrifuged at 5000×g for 10 minutes and resuspended in 0.2 mL PBS. Emission spectra may be collected using an excitation wavelength of 400 nm. Total “brightness” may be corrected to number of cells present in the culture via the OD600 of each culture diluted one thousand-fold (or until OD600=0.5-1.0).

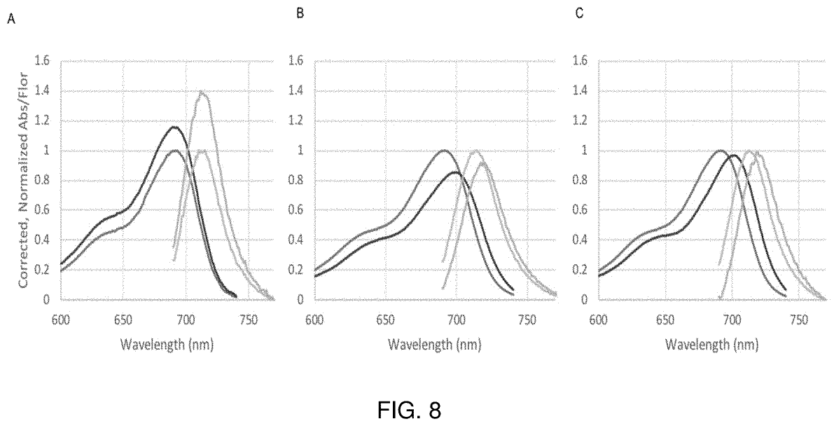

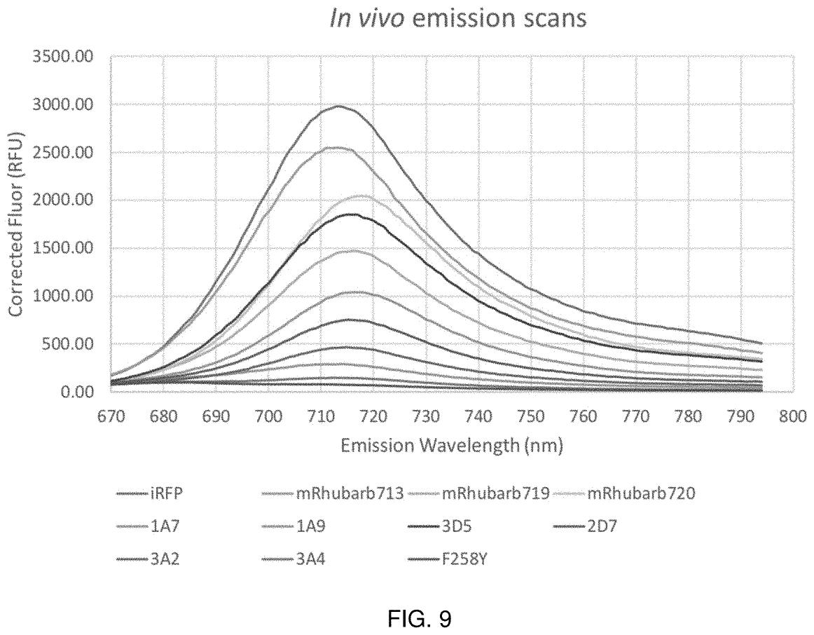

FIG. 8 illustrates tables with excitation (dark colors) and emission (light colors) spectra for iRFP (blue) versus (A) mRhubarb713 (red), (B) mRhubarb719 (red), and (C) mRhubarb720 (red) according to an embodiment of the present invention. All values can be normalized to the original spectra using relative maximum and minimum values as 1 and 0. Brightness may be corrected to iRFP. FIG. 9 illustrates a table with emission spectra for all red-shifted or brighter mutants of iRFP in BL21 E. coli according to an embodiment of the present invention. Fluorescence was corrected to the number of cells present based on the relative OD600 to iRFP cultures. All samples were excited at 400 nm.

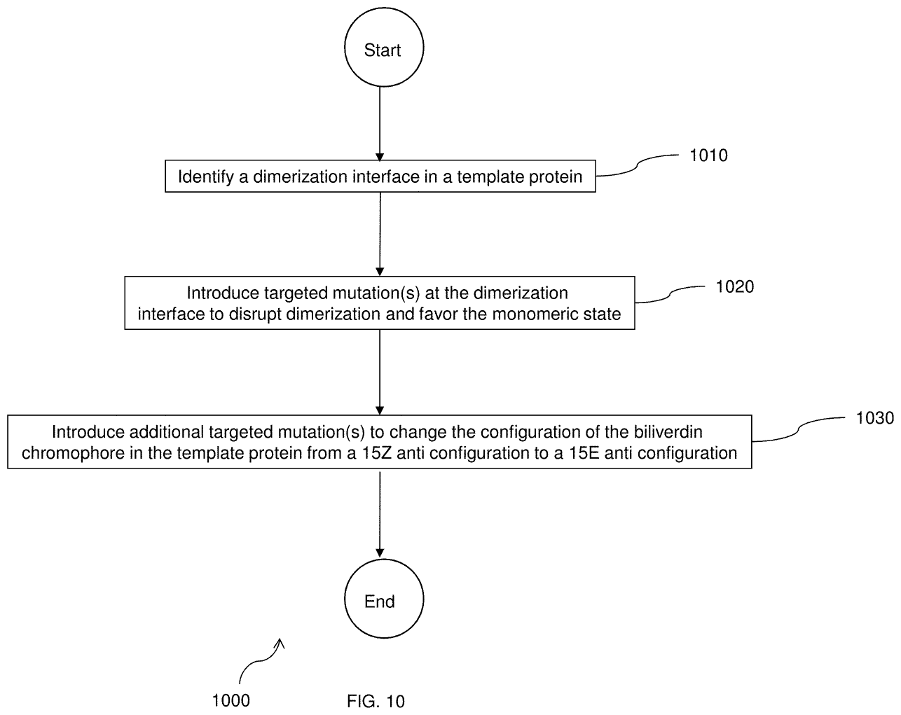

FIG. 10 is a flow diagram illustrating a method 1000 for the engineering of monomeric, red-shifted, and brighter variants of iRFP using structure-guided multi-site mutagenesis according to an embodiment of the present invention. The method 1000 can be used to convert a fluorescent protein into a modified fluorescent protein in a monomeric state. A dimerization interface in a template protein is identified 1010, where the template protein is in a homodimer state. The template protein can be an RpBphP2-derived protein, for example, an iRFP.

One or more targeted mutations are introduced at the dimerization interface to disrupt dimerization and favor the monomeric state 1020. The targeted mutation(s) can be introduced at an F131 position, an F132 position, a Q298 position, a W309 position, and/or a Q310 position in an RpBphP2-derived protein. The introduction of the targeted mutation(s) can involve an isomerization process about the C15=C16 double bond between rings C and D of the biliverdin chromophore.

In at least one embodiment of the invention, one or more additional targeted mutations are introduced to change the configuration of the biliverdin chromophore in the template protein from a 15Z anti configuration to a 15E anti configuration 1030. This can include stabilizing the biliverdin chromophore in a far-red absorbing Pfr state or shifting the biliverdin chromophore towards a far-red absorbing Pfr state.

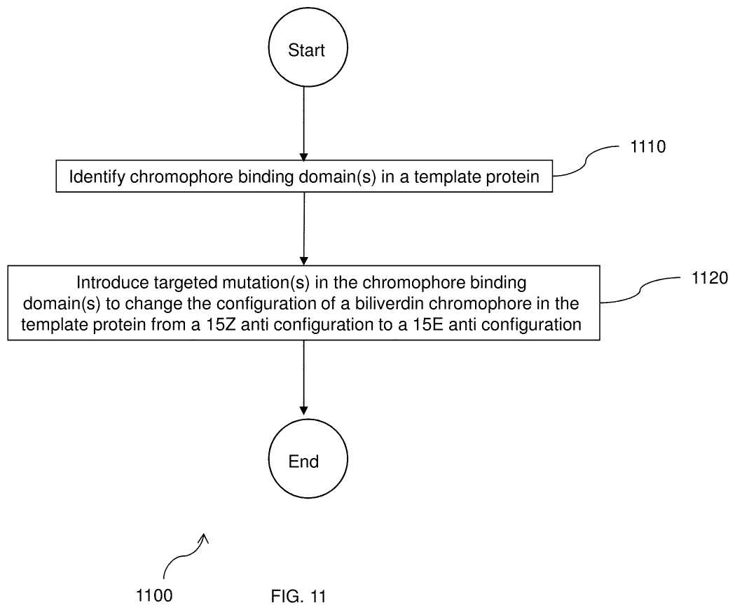

FIG. 11 is a flow diagram illustrating a method 1100 for the engineering of monomeric, red-shifted, and brighter variants of iRFP using structure-guided multi-site mutagenesis according to another embodiment of the present invention. One or more chromophore binding domains can be identified in a template protein 1110. In at least one embodiment, the template protein is an iRFP.

One or more targeted mutations can be introduced in the chromophore binding domain(s) to change the configuration of a biliverdin chromophore in the template protein from a 15Z anti configuration to a 15E anti configuration 1120. The targeted mutation can be introduced at a V6 position, an A7 position, an R8 position, a Q9 position, a P10 position, a Y171 position, a Y173 position, a K193 position, an L196 position, a Y198 position, a T202 position, a V203 position, an R217 position, an R249 position, an F258 position, a T267 position, an 5269 position, an L281 position, a V283 position, an H285 position, an R466 position, an R462 position, and/or an 5468 position in an RpBphP2-derived protein. The introduction of the targeted mutation(s) can stabilize the biliverdin chromophore in a far-red absorbing Pfr state. In addition, the introduction of the targeted mutation(s) can involve an isomerization process about a C15=C16 double bond between rings C and D of the biliverdin chromophore.

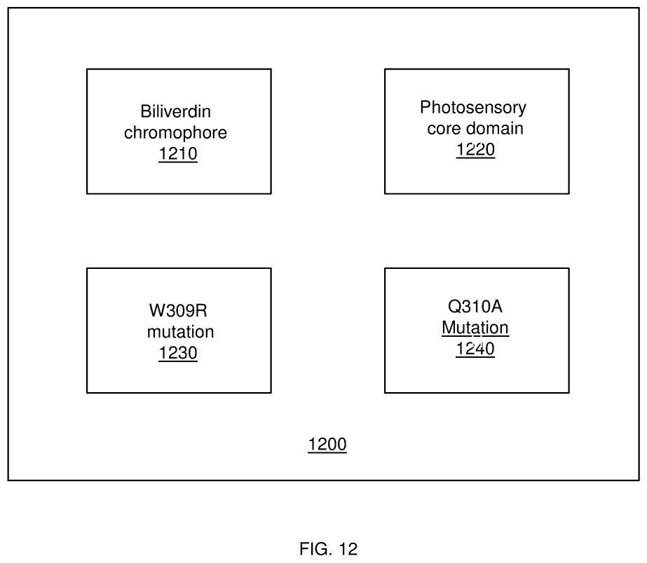

FIG. 12 illustrates a monomeric protein 1200 according to an embodiment of the present invention. The monomeric protein 1200 can include a biliverdin chromophore 1210 and a photosensory core domain (PCD) 1220. The PCD can include a chromophore binding domain (CBD), where the CBD can include a PAS (Per-Arnt-Sim) domain and a GAF (cGMP phosphodiesterase/adenylyl cyclase/FhlA) domain. The monomeric protein 1200 can also include a W309R mutation 1230 and a Q310A mutation 1240. The monomeric protein 1200 may be a variant of iRFP and have a 15-40% increased quantum yield compared to iRFP and a 20-60% increased molecular brightness compared to iRFP.

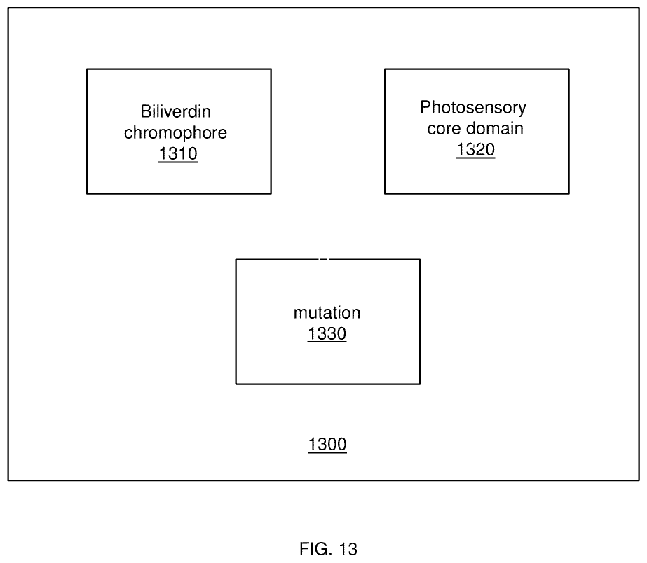

FIG. 13 illustrates a monomeric protein 1300 according to an embodiment of the invention, wherein the monomeric protein 1300 includes a biliverdin chromophore 1310, a photosensory core domain (PCD) 1320, and a mutation 1330. The PCD 1320 can include a chromophore binding domain (CBD) that has a PAS (Per-Arnt-Sim) domain and a GAF (cGMP phosphodiesterase/adenylyl cyclase/FhlA) domain. The mutation 1330 can include a Y198S mutation, a T202Y mutation, a V203I mutation, a W309R mutation, a Q310A mutation, and/or a R134H mutation. The monomeric protein 1300 may be a variant of iRFP and have a 5-20% increased quantum yield compared to iRFP. The monomeric protein 1300 can also have a 6 nm red-shifted emission peak compared to iRFP, an excitation peak of 700 nm, and/or an emission peak of 719 nm.

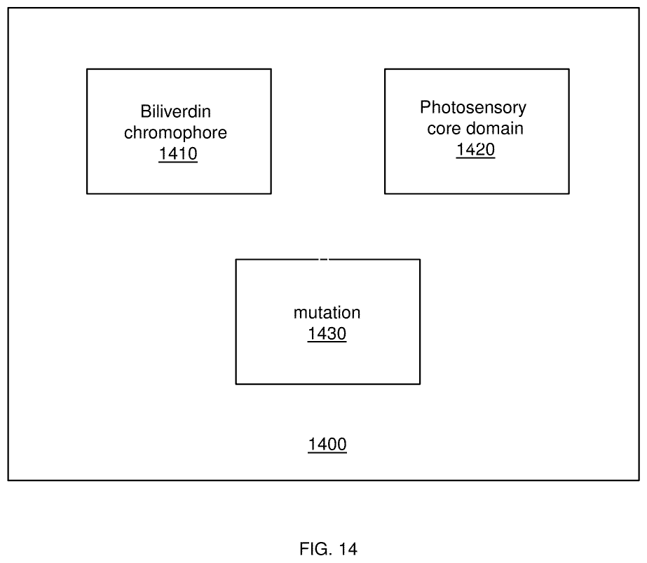

FIG. 14 illustrates a protein 1400 according to an embodiment of the invention, where the protein 1400 includes a biliverdin chromophore 1410, a photosensory core domain (PCD) 1420, and a mutation 1430. The PCD 1420 can include a chromophore binding domain (CBD) having a PAS (Per-Arnt-Sim) domain and a GAF (cGMP phosphodiesterase/adenylyl cyclase/FhlA) domain. The mutation 1430 can include a L196Q mutation, a T202D mutation, a V203I mutation, a W309R mutation, a Q310A mutation, and/or a R134H mutation. The protein 1400 can be a variant of iRFP having a 1-15% increased quantum yield compared to iRFP. In addition, the protein 1400 can have a 7 nm red-shifted emission peak compared to iRFP, an excitation peak of 701 nm, and/or an emission peak of 720 nm.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of at least one other feature, integer, step, operation, element, component, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

### REFERENCES

1. Day, R. N. & Davidson, M. W. The fluorescent protein palette: tools for cellular imaging. Chem Soc Rev 38, 2887-2921 (2009).
2. Yang, M. et al. Whole-body optical imaging of green fluorescent protein-expressing tumors and metastases. Proc. Natl. Acad. Sci. U.S.A. 97, 1206-1211 (2000).
3. Kim, G. et al. Deep-brain imaging via epi-fluorescence Computational Cannula Microscopy. Sci Rep 7, 44791 (2017).
4. Khullar, O., Frangioni, J. V., Grinstaff, M. & Colson, Y. L. Image-guided sentinel lymph node mapping and nanotechnology-based nodal treatment in lung cancer using invisible near-infrared fluorescent light. Semin. Thorac. Cardiovasc. Surg. 21, 309-315 (2009).
5. Yang, X., Kuk, J. & Moffat, K. Conformational differences between the Pfr and Pr states in Pseudomonas aeruginosa bacteriophytochrome. Proc. Natl. Acad. Sci. U.S.A. 106, 15639-15644 (2009).
6. Filonov, G. S. et al. Bright and stable near-infrared fluorescent protein for in vivo imaging. Nat Biotech 29, 757-761 (2011).
7. Shcherbakova, D. M. & Verkhusha, V. V. Near-infrared fluorescent proteins for multicolor in vivo imaging. Nat Meth 10, 751-754 (2013).
8. Campbell, R. E. et al. A monomeric red fluorescent protein. Proceedings of the National Academy of Sciences 99, 7877-7882 (2002).
9. Samma, A. A., Johnson, C. K., Song, S., Alvarez, S. & Zimmer, M. On the origin of fluorescence in bacteriophytochrome infrared fluorescent proteins. J Phys Chem B 114, 15362-15369 (2010).
10. Rockwell, N. C., Shang, L., Martin, S. S. & Lagarias, J. C. Distinct classes of red/far-red photochemistry within the phytochrome superfamily. Proceedings of the National Academy of Sciences 106, 6123-6127 (2009).
11. Yang, X., Kuk, J. & Moffat, K. Crystal structure of Pseudomonas aeruginosa bacteriophytochrome: Photoconversion and signal transduction. Proceedings of the National Academy of Sciences 105, 14715-14720 (2008).
12. Tonikian, R., Zhang, Y., Boone, C. & Sidhu, S. S. Identifying specificity profiles for peptide recognition modules from phage-displayed peptide libraries. Nature Protocols 2, 1368-1386 (2007).
13. Firnberg, E. & Ostermeier, M. PFunkel: efficient, expansive, user-defined mutagenesis. PLoS ONE 7, e52031 (2012).
14. Wall, K. P., Dillon, R. & Knowles, M. K. Fluorescence quantum yield measurements of fluorescent proteins: a laboratory experiment for a biochemistry or molecular biophysics laboratory course. Biochem Mol Biol Educ 43, 52-59 (2015).

1.-42. (canceled)

43. A monomeric protein comprising:
a biliverdin chromophore;
a photosensory core domain (PCD) including a chromophore binding domain (CBD), the CBD including a PAS (Per-Arnt-Sim) domain and a GAF (cGMP phosphodiesterase/adenylyl cyclase/FhlA) domain; and
a mutation.

44. The monomeric protein according to claim 43, wherein said mutation comprises a Y198S mutation.

45. The monomeric protein according to claim 43, wherein said mutation comprises a T202Y mutation.

46. The monomeric protein according to claim 43, wherein said mutation comprises a V203I mutation.

47. The monomeric protein according to claim 43, wherein said mutation comprises a W309R mutation.

48. The monomeric protein according to claim 43, wherein said mutation comprises a Q310A mutation.

49. The monomeric protein according to claim 43, further comprising a R134H mutation.

50. The monomeric protein according to claim 43, wherein said monomeric protein is a variant of iRFP, and wherein said monomeric protein has a 5-20% increased quantum yield compared to iRFP.

51. The monomeric protein according to claim 43, wherein said monomeric protein is a variant of iRFP, and wherein said monomeric protein has a 6 nm red-shifted emission peak compared to iRFP.

52. The monomeric protein according to claim 43, wherein said monomeric includes an excitation peak of 700 nm and an emission peak of 719 nm.

53. A protein comprising
a biliverdin chromophore;
a photosensory core domain (PCD) including a chromophore binding domain (CBD), the CBD including a PAS (Per-Arnt-Sim) domain and a GAF (cGMP phosphodiesterase/adenylyl cyclase/FhlA) domain; and
a mutation.

54. The protein according to claim 53, wherein said mutation comprises a L196Q mutation.

55. The protein according to claim 53, wherein said mutation comprises a T202D mutation.

56. The protein according to claim 53, wherein said mutation comprises a V203I mutation.

57. The protein according to claim 53, wherein said mutation comprises a W309R mutation.

58. The protein according to claim 53, wherein said mutation comprises a Q310A mutation.

59. The protein according to claim 53, further comprising a R134H mutation.

60. The protein according to claim 53, wherein said protein is a variant of iRFP, and wherein said monomeric protein has a 1-15% increased quantum yield compared to iRFP.

61. The protein according to claim 53, wherein said protein is a variant of iRFP, and wherein said protein has a 7 nm red-shifted emission peak compared to iRFP.

62. The protein according to claim 53, wherein said protein includes an excitation peak of 701 nm and an emission peak of 720 nm.
